(19) United States
(12) Patent Application Publication
(10) Pub. No.: US 2003/0013676 A1
(43) Pub. Date: Jan. 16, 2003

(54) Method and composition for prolonging the residence time of drugs in the gut

(76) Inventors: John A. Oaks, Madison, WI (US); Paul Bass, Madison, WI (US); K. Dubear Kroening, Greenville, WI (US)

Correspondence Address: DEWITT ROSS & STEVENS, S.C., Firstar Financial Centre, Suite 401, 8000 Excelsior Drive, Madison, WI, 53717-1914, US

(21) Appl. No.: 10/186,486

(22) Filed: Jul. 1, 2002

Related U.S. Application Data
(60) Provisional application No. 60/302,502, filed on Jul. 2, 2001.

(51) Int. Cl. A61K 31/7076; A61K 31/675

(52) U.S. Cl. 514/48; 514/81

(57) ABSTRACT

The present invention provides methods and compositions for slowing the transit time of pharmaceutical compounds, nutritional supplements, and vitamins through the gastrointestinal tract, prolonging residence time of such compounds, and thereby increasing absorption in the small intestine, by utilizing the cellular regulatory compound cyclic GMP. The present invention also provides methods and compositions for enhancing the bioavailability and therapeutic effectiveness of pharmacologically active agents, vitamins, and nutritional supplements.

### CROSS-REFERENCE TO RELATED APPLICATIONS

This application claims priority under 35 U.S.C. §119(e) to provisional U.S. Patent Application Ser. No. 60/302,502, filed Jul. 2, 2001, which is incorporated by reference herein.

### Federal Government Support

This invention was made with United States government support awarded by the following agencies. NIH A1433007. The United States has certain rights to this invention.

### REFERENCE TO CITATIONS

Complete bibliographical citations to the references can be found in the list preceding the

### FIELD OF THE INVENTION

The present invention generally concerns methods and formulaic compositions of pharmaceutical compounds, vitamins, and nutritional supplements used to prolong transit through the gastrointestinal tract of humans and other animals, thereby increasing absorption. For purposes of the present invention and unless otherwise noted, the term “supplement” will refer collectively to pharmaceutical compounds, vitamins, nutritional supplements, and/or drugs.

### BACKGROUND OF THE INVENTION

The gastrointestinal tract processes and absorbs food, as well as supplements. Compounds travel from the stomach, which stores and digests food and supplements, to the small intestine, which comprises three sections: the duodenum, the jejunum, and the ileum. The small intestine functions to absorb digested food and supplements.

The process of absorbing supplements and food is controlled by a complex system of inhibitory and stimulatory motility mechanisms which are set in motion when compounds are ingested. Specific receptors in the small intestine respond to the specific nutrients ingested, and modulate the transit and absorption rate of compounds. The same factors that affect nutrient absorption influence the intestinal absorption of the supplements. The small intestine has the greatest capacity for absorption of these substances.

For absorption to proceed efficiently, the supplements must arrive at an absorbing surface in a form suitable for absorption, and must remain there long enough in a concentration that enhances absorption. The supplements must then be absorbed by a normal mucosa. Accordingly, considerable advantage would be obtained if a supplement dosage could be retained for a longer period of time within the small intestine for absorption to occur. The period of time during which drugs are in contact with the small intestine is crucial for the efficacy of absorption. Therefore, a reduction of motility rate and transit time will ensure optimal utilization of the absorptive surface.

Absorption of supplements in the small intestine is a function of the molecular structure and composition of the supplement itself, the small intestine's response to the supplement, and to the overall transit time through the small intestine. To the pharmaceutical industry, the rate of passage through the small intestine is of great significance because it affects the quantity of the drug absorbed. For example, in some cases only 1% of pharmaceutical compositions, even drugs for serious medical conditions, are absorbed by the intestine. If the transit of the compound could be slowed down, such that just 1% more was absorbed, the total drug absorbed would double, thereby improving therapeutic efficiency.

Several previous attempts to alter small intestinal transit times have either not been successful (Khosla and Davis, 1987; Davis et al. 1986), or have focused on malabsorption of fatty acids in patients with gastrointestinal conditions for the purposes of nutrition and weight gain (U.S. Pat. Nos. 5,977,175 and 5,817,641). Attempts to alter intestinal motility patterns using invasive nematode parasites have also been made in rats (Castro, 1989). Additionally, lumen-dwelling non-mucosal non-invasive organisms have been found to slow absorption and motility in the gut of the host (Dwinell et al., 1998). However, no practical application of these findings for either of these parasites has been successfully adapted to improving drug, vitamin, and nutritional supplement absorption in humans. Thus, a need exists for specifically optimizing the bioavailability of ingested drug compounds in the small intestine, to improve the overall efficacy of numerous pharmaceutical, supplemental, and nutritional compositions.

The tapeworm Hymenolepis diminuta (H. diminuta) is a chronic parasite of the rat residing within the lumen of the small intestine and migrating along the lumen in a diurnal fashion, corresponding to host food intake (Bråaten and Hopkins, 1969; Read and Kilejian, 1969; Hopkins, 1970). Although H. diminuta secretes a number of small molecules, proteins and glycolipids (Pappas and Read, 1972a; Knowles and Oaks, 1979; Uglem and Just, 1983; Zavras and Roberts, 1985; Oaks and Holy, 1994), this tapeworm is not associated with obvious harmful effects to its rat host (Insler and Roberts, 1976). Some of these secretions regulate physiological processes of the tapeworm such as growth (Cook and Roberts, 1991). Still other secretions inactivate host physiological processes, such as digestive enzyme activity (Pappas and Read, 1972a, b; Uglem and Just, 1983; Pappas and Uglem, 1990). Dwinell et al. (1998) postulated that a secretion from this tapeworm is capable of altering host enteric smooth muscle contractions.

In the uninfected rat and other vertebrate species, two patterns of electrical activity are present in the smooth muscle of its small intestine. One is the digestive pattern of myoelectrical activity that occurs after nutrient ingestion and is characterized by random electrical spiking throughout the length of the small intestine. The second pattern of myoelectric activity, termed the Migrating Myoelectric Complex (MMC), is present in the interdigestive state (Szurszewski, 1969; Carlson et al., 1978). The MMC is divided into 3 phases: Phase I is a period of myoelectric quiescence, followed sequentially by Phase II, a period of irregular spiking activity, and Phase III, a period of maximum myoelectric spiking frequency and amplitude. Phase III is the electrical correlate of smooth muscle contraction with the closing of the intestinal lumen. Because Phase III migrates caudally, it causes the propulsion of the lumenal contents to the colon. In the rat, a complete cycle of the MMC occurs approximately every 15 minutes. As a result, the MMC serves as the “housekeeper” of the small intestine, sweeping the remnants of the preceding meal, as well as any bacteria present in the lumen, toward the caecum and colon.

Parasitic infection can disrupt the MMC and induce a repertoire of myoelectric alterations characteristic of the specific parasite (Palmer et al., 1984; Berry et al, 1986; winell et al., 1994, Palmer and Greenwood-Van Meerveld, 2001). In the case of the tapeworm H. diminuta, there are two characteristic alterations of myoclectric activity, the Repetitive Burst of Action Potential (RBAP) and the Sustained Spike Potential (SSP). Homogenate fractions of whole tapeworms infused into the small intestinal lumen were shown to alter myoelectric activity by inducing RBAP and SSP indistinguishable from those induced by tapeworm infection (Dwinell et al., 1998). These myoelectric patterns induced in the presence of the tapeworm slow movement of contents within the lumen of the intestine (Dwinell et al., 1997). This observation indicated that the physical presence of the tapeworm was not inducing altered myoelectric patterns (Dwinell et al., 1998), but some constituent present in the tapeworm was activating these myoelectric patterns in vivo. Culture medium used previously to maintain H. diminuta in vitro, known as Tapeworm-Conditioned Medium (“TCM”), induces SSP demonstrating that the tapeworm secretes compounds to its surroundings that cause changes in intestinal motility (Kroening et al., 2002).

### SUMMARY OF THE INVENTION

The present invention provides methods and compositions for slowing the transit time of pharmaceutical, vitamin, and supplemental compounds through the gastrointestinal (GI) tract, prolonging residence time of such compounds, and increasing absorption in the small intestine. The present invention also provides methods and compositions for enhancing the bioavailability and therapeutic effectiveness of pharmacologically active agents, as well as vitamins and nutritional supplements.

Accordingly, the present invention provides pharmaceutical, vitamin, or nutritional supplement compositions that can be used in the form of a solid, a solution, an emulsion, a dispersion, and the like, wherein the resulting composition contains the compound of the present invention, as an active ingredient, in a mixture with an organic or inorganic carrier or excipient.

In addition, the present invention is directed to a method for prolonging the residence time of an administered substance in the small intestine of a subject. The method comprises administering to a subject in need of the substance a composition comprising a carrier and cGMP in an amount and form effective to promote contact of the cGMP with the subject's small intestine. The administration prolongs the residence time of the administered substance to assist in the dissolution, bioavailability and/or increased substance absorption through the small intestine.

Further, the invention is directed to a method of enhancing the absorption of orally administered pharmaceuticals, vitamins, and/or supplements. The method comprises administering to a patient a composition comprising a carrier and a dispersion consisting of cGMP, in a form effective to promote the contact of the cGMP with the small intestine, slow the intestinal transit and thereby prolong the residence time and enhance the absorption of orally administered pharmaceuticals, vitamins, and/or nutritional supplements in the small intestine.

The presention invention is also directed to a method of enhancing the bioavailability of an orally ingested pharmaceutical, vitamin, or nutritional supplement. The method comprises administering to a subject, a composition comprising cGMP in an amount and in a form effective for promoting the contact of the cGMP with the small intestine, prolonging residence time, and promoting absorption/bioavailability of the pharmaceutical, vitamin, or nutritional supplement.

Further still, the present invention is directed to a composition useful in prolonging the residence time of an administered substance in the small intestine of a subject, comprising a carrier and cGMP in an amount and form effective to promote contact of the cGMP with the subject's small intestine, thereby prolonging the residence time of the administered substance to assist in the dissolution, bioavailability and/or increased substance absorption through the small intestine.

The instant invention solves the problem of limited absorption thereby improving the bioavailability of a given pharmaceutical, vitamin, or nutritional compound. The methods of this invention provide a means to increase residence time of the compound in the gut. Additionally, to improve supplement absorption in the small intestine, the present invention provides a method for prolonging the GI residence time, which will allow compounds in any dosage form to be more completely dissolved and absorbed.

Further advantages of the invention will appear from a complete review of the Drawings and the Detailed Description, below

### BRIEF DESCRIPTION OF THE DRAWINGS

FIG. 1 is a graph depicting the myoelectric patterns of the small intestine of rats tested in Experiment 1.

FIG. 2 is a bar chart illustrating the effect of lumenal cGMP dose on the induction of Sustained Spike Potentials (SSP) in Experiment 1.

FIG. 3 is a bar chart illustrating the effect of various substances infused into the intestinal lumen of rats on the frequency of SSP in Experiment 1.

### DETAILED DESCRIPTION OF THE INVENTION

The secretory product of the tapeworm, H. diminuta, the intra-cellular regulatory agent guanosine cyclic 3′, 5′-hydrogen phosphate (“cyclic GMP” or “cGMP”), has been determined by the inventors to alter normal intestinal activity and thus slow the motility of pharmaceutical compositions, vitamins, and nutritional supplements through the gut. Other forms of cGMP, as listed in Budavari et al., Eds. (1980), include cyclic guanosine 3′, 5′-cyclic monophosphate; guanosine 3′, 5′-monophosphate; 3′, 5′-GMP; cGMP; guanosine 3′, 5′-(hydrogen phosphate); guanosine 3′, 5′-cyclic monophosphate; and guanosine 3′, 5′-cyclic phosphate.

Additionally, the general category ‘cGMP’ as referred to in the instant application shall include any or all of the additional compounds that may result from intestinal enzymatic alteration including: (1) the dephosphorylated ribonucleotide: riboguanosine or guanosine or deoxyriboguanosine or deoxyguanosine; (2) the other phosphorylated forms of cGMP: guanylate monophosphate or riboguanylate monophosphate or ribodeoxyguanylate monophosphate or deoxyriboguanylate monophosphate. These phosphorylated forms can occur as the 5′-monophosphate, the 2′-monophosphate, the 3′-monophosphate and the 2′, 3′-monophosphate intermediate form; (3) the hydroxylated or deoxy- forms of the ribose sugar of the nucleotide: ribose, deoxyribose, ribose monophosphate or deoxyribose monophosphate; (4) the purine: guanine; (5) the methylated form of guanine: N2-methylguanine or N7-methylguanine; or (6) GMP's metabolic end products: xanthine and uric acid.

The pharmaceutical industry has published a great deal of information on the absorption time for individual pharmacologically active agents and compounds. Such information is found in the numerous pharmacological publications which are readily available to those skilled in the art. For example, if the in vitro model for absorption and release of an agent is 1.5 hours, then the small intestinal residence time for optimal absorption of the agent would be at least 1.5 hours. Thus for pharmacologically active agents, the appropriate residence time is dependent on the time for release of the active agent.

Vitamins and nutritional supplements are absorbed in much the same way food molecules are absorbed, and times for absorption of these compounds should be similar to absorption times for foods containing similar vitamins and minerals.

As used herein, “digestion” encompasses the process of breaking down large molecules into their smaller component molecules, and “absorption” encompasses the transport of a substance from the intestinal lumen through the barrier of the mucosal epithelial cells into the blood and/or lymphatic systems.

### Active Agent

In order to improve the efficacy of pharmaceutical agents, vitamins, and nutritional supplements, residence time must be increased to enhance absorption. One means of increasing absorption of a drug, vitamin, or a nutritional supplement is to alter the contractility of the gut using the cellular regulatory agent cGMP. The tapeworm, H. diminuta, can be made to produce cGMP during in vitro incubation. cGMP can then be isolated from the medium the worm is growing in and used in formulation (Zavras & Roberts, 1985). Ishikawa et al. (1969) determined that cGMP is naturally present in the small intestine of mammals. Dwinell et al., (1997) correlated SSP frequency with increased residence time. However, as illustrated in the experiments below, cGMP significantly alters the motility of the gut, thereby increasing the residence time of substances therein.

### Carriers

The active ingredient may be compounded with the usual nontoxic, pharmaceutically acceptable carriers for tablets, capsules, solutions, emulsions, suspensions, and any other form suitable for use and known to the art. These carriers may include any carrier suitable for use in manufacturing preparations of pharmaceuticals, supplements, or vitamins, in solid, semisolid, or liquid form. In addition, emulsifying, auxiliary, stabilizing, thickening, and coloring agents may be used. For example, gum acacia, gum agar, sodium alginate, bentonite and powdered cellulose can be used.

cGMP is included in the pharmaceutical composition in an amount sufficient to produce the desired effect of altering motility in the gut. Pharmaceutical, vitamin, or supplement compositions containing the cGMP compound may be in any form suitable for oral use including lozenges, hard gelatin caplets, soft gelatin caplets, tablets, suspensions, emulsions, and the like. They may also be mixed with inactive materials such as water, oils, paraffins, powders, granules, syrups, detergents, salts, suspending, or with agents for emulsifying, stabilizing, buffering, preserving, coloring, disintegrating, solubilizing, flavoring, sweetening, and the like.

### Dose

The effective dosage depends on a number of factors, including type of supplement, and age and weight of the recipient. Generally, an effective dosage is an amount that is effective to slow GI transit to allow the supplement additional time to be absorbed. One of ordinary skill in the art should be able to readily determine the optimum dosage, the procedure of dosage and the number of doses per day. In use, the composition, encapsulated or not, is typically ingested orally either prior to or along with the supplement to promote increased time in the lumen for adsorption of the supplement.

In the preferred embodiment of the present invention, the pharmaceutical, vitamin, or nutritional supplement article is enterically combined in a suitable form with the cGMP and inactive agents. cGMP will produce a prolonged transit time in the small intestine, the active drug, vitamin, or nutritional compound will be present in the small intestine over a longer period of time, thus increasing the absorption.

### Experiments

The following experiments are included solely to aid in a more complete understanding of the subject invention. The experiments do not limit the scope of the invention described herein in any fashion.

In the present studies, the hypothesis that H. diminuta secretes myoelectric (motility)-altering compounds into in vitro culture was tested. Those endogenous compounds of the parasite possessing the ability to alter intestinal physiology are referred to as signal factors. Secretion of these signal factors from the tapeworm is necessary to induce altered enteric smooth muscle activity.

### Experiment 1

The aim of this experiment was to evaluate cGMP as a potential endogenous substance involved in those afferent neuro-sensory pathways that might mediate tapeworm-induced changes in small intestinal smooth muscle contractility. Exogenous intraduodenally administered cGMP, possibly acting on various receptor types, was found to mimic the SSP pattern generated by a tapeworm infection in the rat. These novel observations increased the understanding of how intralumenal signal molecules associated with strictly lumenal parasites interact with neuro-pathways in host regulatory systems to activate a repertoire of intestinal pathophysiological responses.

### Animal Preparation for Signal Molecule Bioassay

Outbred male rats (Sprague Dawley, Harlan Sprague Dawley, Inc., Indianapolis, Ind.) used in this study were housed singly and maintained on a 12:12 hour light:dark regime. All rats used in this bioassay procedure were uninfected. The design and surgical implantation procedure of the intestinal extracellular bipolar electrodes was described previously (Dwinell et al., 1994, 1997). In brief, 4 bipolar electrodes were surgically sutured to the intestinal serosa of each rat according to the methods of Dwinell et al (1994), which is incorporated herein by reference for a description of the materials and methods of this experiment. Three electrodes (J1-J3) were implanted on the jejunum at 10 cm intervals, placing the first electrode (J1) 10 cm caudal from the ligament of Treitz. The fourth electrode (J4) was placed 20 cm orad from the ileo-caecal junction. In addition, a cannula was implanted with one end residing in the lumen of the mid-duodenum, whereas the other end was exteriorized to allow infusion of test fractions.

All compounds tested were infused into the duodenum via the cannula. Compounds were delivered in 0.2 ml aliquots followed immediately by a 0.2 ml saline cannula rinse. These volumes were used in order to prevent muscle contraction due to stretch from larger bolus volumes. To observe the induction of SSP, intestinal myoelectric activity was recorded for 90 minutes following infusion.

Consistent with the protocol of Dwinell et al. (1994), intestinal myoelectric activity was not recorded for the first 5 days after implantation surgery. Control recordings were taken after this period to assure the return of normal myoelectric patterns of the MMC following the cessation of post-surgical ileus. Periodically, “control” recordings were made with saline on the intervening days between the tests with TCM or its fractions.

For intragastric infusion, rats (n=4) were lightly sedated with Halothane in order to insert a gastric tube per os and infused with 0.3ml of 10 mM cGMP in saline directly into the lumen of the stomach. Thirty minutes of control myoelectric recording was always performed before infusion of any substance. Five to seven minutes after infusion, the rats were reconnected to the recorder. Effects of handling and anesthesia were not evident on intestinal motility, since on their reconnection to the recorder, all rats showed normal intestinal myoelectric activity. This procedure was repeated with the same rats on a different day with 0.3 ml 100 mM cGMP (equal to 100× the minimal dose required to increase SSP frequency by infusion into the lumen of the small intestine) and on a separate day with 0.3 ml saline as a vehicle control. Post-oral dosing myoelectric recordings were 90 minutes in duration.

### Preparation of Tapeworm Culture Medium

Tapeworms, used for in vitro culture, were collected 20-40 days after infection by flushing rat small intestine with room temperature (22° C.) Krebs-Ringer's-Tris Maleate buffer (KRTM, pH 7.2). Tapeworms of this age were selected because Dwinell et al. (1994) demonstrated that maximum altered myoelectric activity did not occur until at least 10 days after infection. All tapeworms used for in vitro culture were from 35 cysticercoid infections per rat and all tapeworms transferred to culture were visually intact. The tapeworms were rinsed twice in KRTM and then twice more in sterile Roswell Park Memorial Institute (RPMI) 1640 medium (Fisher Scientific, Chicago, Ill.) before being placed in culture.

To obtain TCM, 5 tapeworms were put into 50 ml of sterile RPMI 1640 (pH 7.5) containing 25 mM N-[2-hydroxyethyl] piperazine-N′-[2-ethanesulfonic acid] (AEPES), 100 U/ml penicillin, and 0.1 mg/ml streptomycin (Sigma Co., St. Louis, Mo.) The culture flasks with loosened caps were placed in a static tissue culture incubator (Forma Scientific, Marietta, Ohio) at 37° C., 80% humidity, 5% CO₂/air, and then cultured overnight (approximately 12 hours).

To collect TCM, tapeworms were removed from the culture flasks with a sterile hook. However, before removing the tapeworms from the culture flasks, the color of the neutral red pH indicator was checked to determine that the pH was not below 6.8 and all tapeworms were visually inspected to insure that they were motile and intact at the end of the culture period. During these experiments, no autolysis or broken tapeworms were observed, nor was the pH of the TCM below pH 6.8 after overnight in vitro culture.

### Treatment of Tapeworm-Conditioned Medium

To partially characterize the signal factor(s) responsible for altering myoelectric activity, TCM was processed before bioassay in the following ways:

1. passed through an Amicon DIAFLOW Ultrafiltration PM10 membrane (Millipore Corp., Bedford, Mass.) at 4° C. under pressure to collect molecules smaller than 10,000 MW;

2. frozen up to 6 months at −20° C.;

3. boiled (100° C. for 30 minutes);

4. chloroform extracted;

5. extracted by the methods of Folsch et al. (1957); or

6. proteinase K digested.

After filtration, TCM remaining above the filter as well as the filtered TCM were collected. Treated TCM and control samples were bioassayed for their ability to initiate SSP or RBAP myoelectric activity. Samples bioassayed consisted of the following:

1. Saline (labeled “Saline”),

2. Control RPMI 1640 medium incubated under conditions identical to the preparation of TCM (labeled “RPMI”),

3. TCM removed directly from culture immediately before bioassay (labeled “Fresh TCM”),

4. TCM frozen for 24 hours then thawed (labeled “Frozen TC”),

5. TCM filtrate containing <10,000 MW molecules (labeled “Filtered TCM”),

6. TCM retained under pressure but not allowed to pass through the membrane filter (labeled “Retained TCM”), or

7. an “add back” of 1/1 (v/v) Filtered TC plus Retentate (labeled “Filt +Ret”).

TCM was subjected to lipid extraction for nonpolar lipids by mixing equal volumes of chloroform TCM and centrifuging at 15,000×g for 5 minutes at 4° C. The chloroform layer was removed and extraction of the aqueous layer with chloroform repeated (labeled “Chloroform Ext.”). Additionally, both polar and nonpolar lipids were extracted after the methods of Folch et al. (1957) and reported by Cain et al. (1977). Briefly, equal volumes of TCM and chloroform/methanol (2:1) were mixed. The upper chloroform-containing phase was removed and discarded. An equal volume of chloroform/methanol (2:1) containing MgCl₂ was thoroughly mixed with the retained aqueous phase, centrifuged as before and the aqueous phase (labeled “Folch Wash”) was removed for testing in the rat bioassay.

The TCM was also subjected to proteinase treatment. Proteinase K bound to agarose beads (5 mg/ml; Sigma) was prepared per manufacturer's instructions. The Proteinase K-agarose was rinsed twice in 50 mM HEPES buffer (pH 7.4) and resuspended in 200 μl HEPES buffer (pH 7.4). Then 100 μl of this suspension was added to 900 μl of TCM at 37° C. for 2 hours or overnight. Proteinase K-treated TCM (labeled “Proteinase K”) was then bioassayed as described below. To determine if the signal factor could be denatured, TCM was boiled for 30 minutes (labeled “Boiled TCM”)

### pH and Myoelectric Activity

Mettrick (1971) noted that in H. diminuta infected rats the pH of the small intestine was lowered to 5.5. Because the altered pH environment of the infected intestinal lumen might induce the myoelectric alterations observed in the presence of the tapeworm, RPMI 1640 medium adjusted to both pH 7.4 or 5.5 was tested in the bioassay system.

### Electromyographic Recording Schedule

In vivo intestinal myoelectric activity was recorded with a polygraph chart recorder (Grass Instruments, Quincy, Mass.). The myoelectric signal from each electrode was simultaneously recorded on paper and by a personal computer with an I/O board and WINDAQ software (Dataq Instruments, Akron, Ohio) connected to the polygraph recorder by an analog-to-digital converter (Dataq Instruments).

Food was removed on each recording day at 0800 hour to prevent animal feeding and the subsequent disruption of the interdigestive myoelectric pattern. Recording occurred between 1300 and 1900 hours. All recording sessions for each animal were at least 1.5 hour in duration, and all animals were recorded while awake and unrestrained. Vehicle (0.9% saline or in some cases RPMI1640) was used as control for test substances.

The following compounds were infused individually at the concentrations indicated [* indicates a growth regulating factor identified by Zavras and Roberts, 1984, 1985]: *acetate (50 mM), adenosine 3′, 5′-cyclic phosphate (cAMP; 10 nM-100 mM),*D-glucosaminic acid (25mM), guanine (100 nm-10 mM), guanine monophosphate (10 mM), guanosine (10 mM), *guanosine 3′, 5′-cyclic monphosphate (cGMP; 100 nM-100 mM), lactate (20 mM) and *succinate (100 mM).

The following compounds were infused as a group: alanine (22.45 mM), asparagine (430 nM), aspartic acid (150 nM), glutamic acid (1.36 mM), glycine (1.33 mM), histidine (96.7 nM), isoleucine (381 nM), leucine (381 nM), lysine (274 nM), methionine (101 nM), phenylalanine (90.8 nM), proline (174 nM), serine (285 nM), threonine (168 mM), tyrosine (159 nM), and valine (171 nM).

Two types of control recordings, a baseline and an intermittent control recording, were made with infusion of saline to determine if the electrodes were recording appropriately and to assure that frequency of the normal interdigestive myoelectric patterns observed in these experiments were consistent with those of previous studies (Dwinell et al., 1994, 1998). Five days after electrode and cannula implantation surgery, 3 consecutive baseline 90 minute recordings were made on separate days in each rat to confirm the presence of normal intestinal myoelectric activity at the end of the immediate post surgical period. In addition, intermittent control recordings were made in order to show that myoelectric activity remained normal over the course of the experimental period.

### Results

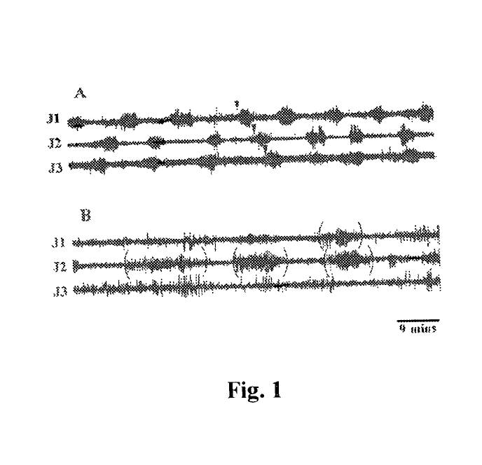

Secreted compounds were infused via the duodenal cannula and the frequency of SSP electrical patterns was determined from recordings of myoelectric activity. FIG. 1 illustrates the myoelectric patterns of the small intestine. The normal interdigestive myoelectric pattern on infusion of saline is shown in Section A. Interdigestive patterns constitute the migrating myoelectric complex (MMC), a series of 3 phases marked by different levels of electrical spiking on the three electrodes, J1, J2 and J3. The third and final phase of the MMC (marked by arrowheads) is a period of >90% spiking and represents a series of contractions migrating caudad along the small intestine. The caudad migration of phase III between electrode sites propels lumenal content toward the caecum. The sustained spike potentials (SSP) on infusion of 10 mM cGMP are shown in Section B. SSP are indicated by brackets on electrodes J1 and J2. SSP represent contractions that close the intestinal lumen, and do not migrate from electrode to electrode. The frequency and lengths of the SSP and the reduction of phase III of the MMC frequency reduces the overall propulsion of lumenal content resulting in a slowing of small intestinal transit.

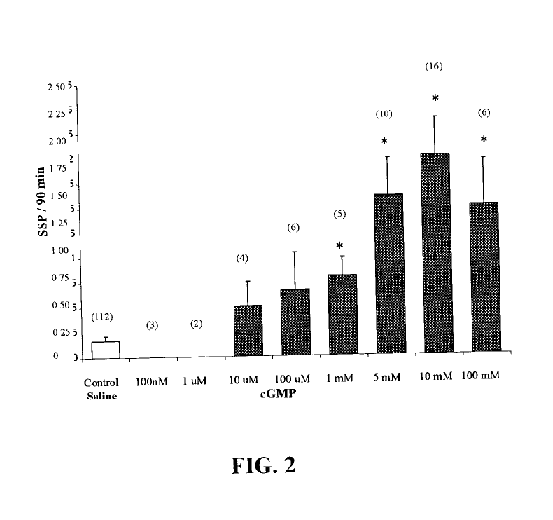

FIG. 2 illustrates the effect of lumenal cGMP dose on the induction of SSP. A significant increase in SSP frequency was seen in a range of 1-100 mM, indicating that SSP response to cGMP is dose dependent. The asterisk (*) indicates that the frequency of SSP is significantly different from the numbers of SSP occurring in response to the saline. Numbers in parentheses are numbers of rats tested. Data were analyzed by the Student t-test. Significance was P≧0.05.

Of the substances tested, only cGMP initiated SSP myoelectric patterns. cGMP activated SSP in a concentration dependent manner. Both the TCM containing secreted cGMP and 10 mM cGMP in physiological saline directly infused into the intestine lose their ability to stimulate SSP when incubated with phosphodiesterase before bioassay.

Cyclic nucleotides were degraded to their 5′-monophosphate derivatives by incubation with bovine brain phosphodiesterase (PDE; cat. # P-0134, Sigma Co., St. Louis, Mo.). One activity unit of PDE (de-esterifies 1 μM cyclic nucleotide/min at 30° C.) was added to 1 μl of 10 mM cGMP or TCM and incubated at 30° C. for 16 hours. The samples were subsequently heated to 100° C. for 3 minutes to destroy PDE activity, allowed to cool to room temperature and then infused (0.2 ml aliquots followed immediately by a 0.2 ml saline cannula rinse) into the small intestinal lumen of uninfected instrumented rats via duodenal the cannula. cGMP specific ELISA (Amersham) determined that PDE treatment of both 10 mM cGMP and TCM, reduced cGMW in both samples to below detectable limits. Recordings made after infusion of a test substance were 90 minutes in length. Control myoelectric recordings were taken both on the days before recording (90 minutes) and on the day of infusion (30 minutes) prior to sample infusion.

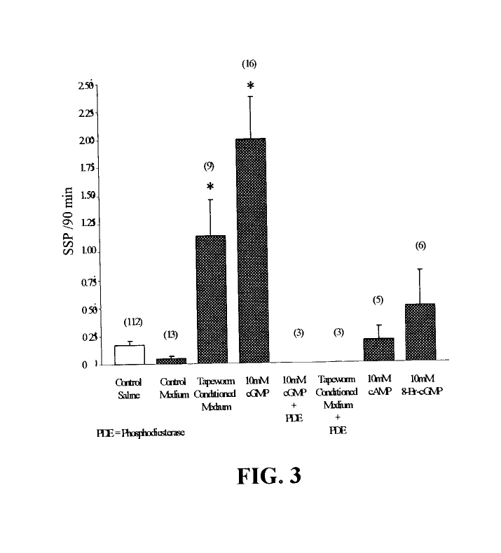

FIG. 3 illustrates the effect of various substances infused (0.2 ml) into the intestinal lumen on the frequency of SSP. Infusion into the intestine of cGMP (10 MM) and tapeworm conditioned medium collected 12 hours after incubations with 5 tapeworms significantly increased the frequency of SSP. 15 day old tapeworms in 50 ml of medium RPMI 1640 significantly increased the frequency of SSP when compared to the infusion of either control saline or control medium (RPMI 1640). However, control values for SSP frequency were also obtained when 10 mM cGMP solution or tapeworm conditioned medium was incubated in phosphodiesterase (PDE) before infusion. SSP frequency response to the infusion of 10 mM or 100 mM cAMP, 10 mM guanine, or 10 mM guanosine was not significantly different from saline controls. Table 1 below shows some of these results and the results of other tested substances.

TABLE 1

| | Number | SSP/90 minutes | |
|---|---|---|---|
| Treatment | of Rats | (Mean ± SE) | p-value |
| Control | 77 | 0.19 ± 0.05 | |
| cAMP | | | |
| 10 nM | 1 | 0 | |
| 1 mM | 2 | 0 | 0.0003 |
| 10 mM (FIG. 3) | 3 | 1.33 ± 0.72 | 0.33 |
| cGMP (FIG. 2) | | | |
| 100 nM | 1 | 0 | |
| 1 μm | 1 | 0 | |
| 10 μm | 2 | 0 | 0.0003 |
| 100 μm | 3 | 1.33 ± 0.54 | 0.23 |
| 1 mM | 2 | 0.50 ± 0.35 | 0.65 |
| 5 mM | 2 | 0.50 ± 0.35 | 0.65 |
| 10 mM | 13 | 2.23 ± 0.44 | 0.0007 |
| 100 mM | 1 | 1 | |
| 0.5M | 1 | 2 | |
| 0.05M cGMP + 0.05 NaAc pH 5.0 | 1 | 0 | |
| NaAc pH 5.0 | 2 | 3.50 ± 1.77 | 0.41 |
| 10 mM cGMP + 1 Unit PDE (FIG. 3) | 2 | 0 | 0.0004 |
| 10X Acid Mixture | 4 | 2.50 ± 1.03 | 0.15 |
| 10 mM Guanosine | 4 | 0.75 ± 0.41 | 0.55 |
| 0.1 mM Guanine | 1 | 2 | |
| 1.0 mM Guanine | 2 | 2.00 ± 1.41 | 0.53 |
| 10 mM Guanine | 2 | 0.50 ± 0.35 | 0.65 |
| 0.25 D-Glucosaminic Acid | 2 | 0 | 0.0004 |

None of the other tapeworm-secreted molecules, including the structurally related purine nucleotide, cAMP, were able to stimulate SSP above background levels as illustrated in FIG. 3. In addition, no response was observed on infusion of the cyclic nucleotides, cUMP and cIMP (data not shown). The cell-permeant cGMP analog, 8-Br-cGMP (0.2 ml of 10 mM) introduced into the intestinal lumen did not significantly increase the SSP frequency above background. These data suggest that the SSP myoelectric pattern is a specific response to the cyclized form of GMP and not a generalized response to purines or to other cyclized nucleotides. In addition, the lack of response to 8-Br-cGMP placed in the intestinal lumen strongly suggests that the receptor for cGMP is on the exterior of the cGMP-responsive cells in the intestine.

Neither cGMP (1.0 ml of 100 mM) injected intraperitoneally nor cGMP (0.3 ml of 10 mM and 100 mM) introduced to the stomach per os initiated the SSP pattern in the small intestine. The lack of intestinal response to the infusion of cGMP into the stomach suggests that if gastric cGMP-responsive cells exist, they are not responsible for the SSP response in the intestine. The responsiveness of the intestine to lumenal infusion of cGMW, but its failure of the intestine to respond to intraperitoneally-injected cGMP indicates that the receptors for cGMP are most likely on the lumenal aspect of the small intestine. The lack of induction of SSP by other cyclic nucleotides strongly suggests that a specific cGMP receptor is involved. Taken together, these data indicate that cGMP secreted by Hymenolepis to the intestinal lumen can serve as a specific extracellular signal molecule regulating host small intestinal motility.

cGMP has been shown to activate the SSP, a unique myoelectric pattern in intestinal smooth muscle that constricts the intestinal lumen for a relatively long duration (6.5-45 min). The location for the cellular transducer of cGMP signaling in the host is unknown, but our data indicate that it is likely displayed on either lumenal epithelial cells or closely associated cells, such as the intrinsic or extrinsic neurons of the enteric nervous system. The evidence shows that slowed intestinal transit occurring as a result of tapeworm infection is the outcome of the cGMP-induced intestinal constriction (SSP) that diminishes transit. This permits the tapeworm to complete its orad circadian migration and to remain in the small intestinal lumen for the life of the host.

### Experiment 2: Rate of Drug Uptake

The purpose of this experiment is to assess the rate of uptake of model drugs by the rat's small intestine treated with transit slowing cGMP. cGMP was shown to be the signal molecule causing changes in the interdigestive smooth muscle contractile patterns and slowing transit in the lumen of the intestine. The contractile pattern caused by cGMP, i.e. SSP, does not migrate down the intestine and replaces propulsive contractile activity for up to 45 minutes. These patterns were originally observed in tapeworm infections of the rat and tapeworm secretions were shown to contain cGMP.

Since uptake of compounds, i.e., drugs and nutrients, from the lumen of the intestine depends upon the length of exposure of these compounds to intestinal uptake mechanisms, the residence time of absorbed compounds in the intestine determines their exposure to uptake mechanisms. Slowing of the passage of compounds through the intestinal lumen increases the residence time for lumenal content, increasing the uptake of absorbed compounds and their subsequent bioavailability.

In brief, model drugs are introduced directly into the lumen of the rat's small intestine and the concentration of these model drugs is measured in the blood over time. The blood values and concentration kinetics of these model drugs are compared after infusion into the intestine alone or when infused with cGMP.

More specifically, a cannula is surgically implanted into the duodenal region of each test rat, extending from the lumen of the duodenum, across the peritoneum, under the skin to the abdominal wall and finally to an exit from the skin at the nape of the neck. A second cannula is installed from the nape of the neck to the neck and inserted into the superior vena cava near the cervical thoracic inlet. During the surgery, both cannulas are filled with sterile saline and plugged with metal pins. Rats are allowed at least 5 days to recover from implantation surgery before any manipulation occurs. All rats are housed individually after surgery to prevent damage to the cannulas. All experimental animals have been eating and drinking freely during the 5 days before the experiment.

On the day of the experiment, food is removed from cages of all animals in the test group, but water is provided ad libitim. Both cannulas are flushed with sterile saline in each experimental rat to assure that all cannulas are open. A 0.1 ml predrug administration sample is taken and a model drug is infused into the duodenum. 0.1 ml blood samples are taken at times: 0, 15, 30, 45, 60 & 75 minutes. Drug concentration is quantified in blood samples and the kinetics of uptake are compared statistically between groups of experimental animals.

Three groups of experimental animals are tested with the following treatments: group #1 receives model drug only via the duodenal cannula; group #2 receives cGMP (0.2 ml of 10 mM, a dose know to activate SSP) at −15, 0, 15 & 30 minutes and drugs will be infused at 0 minutes; and group #3 receives cGMP (0.2 ml of 10 mM) only once, i.e., along with drug at 0 minutes.

The uptakes of two model drugs explored under this experimental plan are: ethylene diamine tetra acetic acid (EDTA) and 4-[2-Hydroxy-3-[(1-methylene)aminol]propoxy]benzeneacetamide (atenolol). These two drugs are chosen because they are poorly absorbed and use different pathways to cross the lumenal epithelium into the intestinal tissues. EDTA depends upon paracellular diffusion and atenolol enters the intestine by transcellular uptake. EDTA is used labeled with the radionucleide, ¹⁴C, and quantified by scintillation counting. Atenolol is quantified via high performance liquid chromatography (HPLC) using electrochemical detection.

Although the invention has been described with reference to the disclosed embodiments, those skilled in the art will recognize that the specific embodiments taught hereinabove are only illustrative of the invention. It should be understood that various modifications can be made without departing from the spirit of the invention.

### Bibliography of Citations

U.S. Pat. No. 5,977,175 to Lin.

U.S. Pat. No. 5,817,641 to Waldman et al.

Berry et al. 1986. Am. J Vet. Res. 47: 27-30.

Bråten and Hopkins. 1969. Parasitology. 59: 891-905.

Bulbring and Crema. 1958. Br. J Pharmacol. 13:444-457.

Budavari et al. (Eds.). 1989. The Merck Index, Merck & Co., Eleventh Edition, p. 422.

Cain et al. 1977. J. of Parasite. 63: 486-491.

Carey et al. 1994. Am. J Physiol. 267:R156-R163.

Carlson et al. 1978. J. Pharmacol. Exp. Ther. 172:367-376.

Castex et al. 1994. Gastroenterology 107:976-984.

Castro. 1989. Parasitol. Today 5:11-19.

Champaneria et al. 1992. Br. J Pharmacol. 106:693-696.

Coelho et al. 1986. J. Surg. Res. 41: 274-278.

Cook and Roberts. 1991. J. Parasitology 77: 21-25.

Davis et al. 1986. Pharm. Res. 3 :208-213.

de Boer et al. 1996. J. Pharmacol. Exp. Ther. 277:852-860.

Dwinell et al. 1994. Am. J Physiol. 267(5 Pt. 1):G851-G858.

Dwinell et al. 1997. Am. J Physiol. 273(2 Pt. 1):G480-G485.

Dwinell et al. 1998. J. Parasitol 84:673-680.

Findlay et al. 1981. J. Immunol. 126: 1728-1730.

Folch, et al. 1957. J. Biol. Chem. 226: 497-509.

Forte. 1999. Regulatory Peptides. 81: 25-39.

Foster and Lee. 1996. Parasitology 112: 97-104.

Graf and Sarna. 1996. Am. J Physiol. 270:G992-G1000.

Grider et al. 1996. Am. J Physiol. 270:G778-G782.

Hopkins. 1970. Parasitology 60: 255-271.

Husebye et al. 1994. Dig. Dis. and Sciences 39: 946-956.

Insler and Roberts. 1976. Exp. Parasit. 39: 351-357.

Ishikawa et al. 1969. J. Bio. Chem. 244:6371-6376.

Jiang et al. 2000. Gastroenterology 119:1267-1275.

Khosla and Davis. 1987. J. Pharm. Pharmacol. 39:47-49.

Kiliaan et al. 1998. Am. J Physiol. 275:G1037-G1044.

Knowles and Oaks. 1979. J. Parasit. 65: 715-731.

Kroening, et al. 2002. J. Parasitol. 88: 227-231

Lin et al. 1997. Dig. Dis. and Sciences 42: 19-25.

Lordal and Hellstrom. 1999. Neurogastroenterol. Motil. 11:1-10.

Lucchelli et al. 2000. Arch. Pharmacol. 362:284-289.

Madara and Dharmsathaphorn. 1985. J. Cell Bio. 101:2124-2133.

Matheis et al. 1980. Am. J Physiol. 239:G382-G386.

McLean and Coupar. 1996. Eur J Pharmacol. 312:215-225.

McKay et al. 1991. Exp. Parasitol. 73:15-26.

Mercer et al. 1987. Mol. & Biochem. Parasit. 26: 225-234.

Mettrick. 1971. Exp. Parasit. 29: 386-401.

Mettrick and Podesta. 1982. Int. J Parasitol. 12: 151-154.

Nash et al. 1987. J. Clinical Investigation 80:1104-1113.

Niwa and Miyazato. 1996. J. Helminthology 70:33-41.

Oaks and Holy. 1994. Exp. Parasit. 79: 292-300.

Ormsbee et al. 1984. J. Pharmacol. Exp. Ther. 231:436-440.

Ovington and Behm. 1997. Memorias do Instituto Oswaldo Cruz 92:93-104.

Palmer et al. 1984. Exp. Parasit. 57: 132-141.

Palmer and Greenwood-Van Meerveld. 2001. J. Parasit. 87: 483-504.

Pappas and Read. 1972a. J. Parasit. 72: 383-391.

Pappas and Read. 1972b. Biological Bulletin 143: 605-616.

Pappas and Read. 1975. Exp. Parasitol. 37: 469-530.

Pappas and Uglem. 1990. Parasitology 101: 455-464.

Parkos. 1997. Bioessays 19:865-873.

Pineiro-Carrero et al. 1991. Am. J. Physiol. 260:G232-239.

Read and Kilejian. 1969. J. Parasit. 55: 574-578.

Ribeiro and Webb. 1983. Int. J Parasit. 13: 101-106.

Ruckebusch and Fioramonti. 1975. Gastroenterology 68:1500-1508.

Sagarda et al. 1990. Life Sci. 46:1207-1216.

Schreiber et al. 1998. Eur. Neuropsycholpharmacol. 8:297-302.

Scudamore et a]. 1995. J. Exp. Med. 182: 1871-1881.

Scott et al. 1988. Am. J Phys.: Gastrointestinal and Liver Physiology 255: G505-G511.

See et al. 1990. Am. J. Physiol. 259:G593-G598.

Szurszewski. 1969. Am. J Phys.:Gastrointestinal and Liver Physiology 217: 1757-1763.

Trendelenburg. 1917. Naunyn-Schmiedegerg 's Arch. Exp. Phath. Pharmak. 81:55-129.

Uglem and Just. 1983. Science 220: 79-81.

Van DerVorst et al. 1990. Annales de Societe Gelge Med Tropicale 70:113-120.

Weisbrodt et al. 1980. J. Pharmacol Exp. Ther. 214:333-338.

Wilson and Wiseman. 1954. J. Physiol. 123:116-125.

Wilson and Baldwin. 1999. Microcirculation 6:189-198.

Yamamoto et al. 1999. Neurogastroenterol Motil 11:457-465.

Zavras and Roberts. 1984. J. Parasitol. 70, 937.

Zavras and Roberts. 1985. J. Parasitol. 70: 937-944 (1985)

Zhao et al. 2000. Am. J Phys.:Gastrointestinal and Liver Physiology 278: G866-870.

What is claimed:

1. A method for prolonging the residence time of an administered substance in the small intestine of a subject, comprising administering to a subject in need of the substance a composition comprising a carrier and cGMP in an amount and form effective to promote contact of the cGMP with the subject's small intestine, thereby prolonging the residence time of the administered substance to assist in the dissolution, bioavailability and/or increased substance absorption through the small intestine.

2. The method of claim 1, wherein the composition is administered orally.

3. The method of claim 1, further comprising administering the composition concurrently with the substance.

4. The method of claim 3, where the administered substance includes one or more of an active pharmaceutical, vitamin, or supplement agent.

5. The method of claim 1, wherein the cGMP is in solid, semi-solid, or liquid form.

6. The method of claim 1, wherein the cGMP is selected from the group consisting of: cyclic guanosine 3′, 5′-cyclic monophosphate; guanosine 3′, 5′-monophosphate; 3′, 5′-GMP; cGMP; guanosine 3′, 5′-(hydrogen phosphate); guanosine 3′, 5′-cyclic monophosphate; and guanosine 3′, 5′-cyclic phosphate.

7. The method of claim 1 wherein the carrier is selected from the group of pharmaceutically acceptable carriers consisting of tablets, capsules, solutions, emulsions and suspensions.

8. A method of enhancing the absorption of orally administered pharmaceuticals, vitamins, and/or supplements, comprising administering to a patient a composition comprising a carrier and a dispersion consisting of cGMP, in a form effective to promote the contact of the cGMP with the small intestine and thereby prolong the residence time and enhance the absorption of orally administered pharmaceuticals, vitamins, and/or nutritional supplements in the small intestine.

9. The method of claim 8, wherein the composition is administered orally.

10. The method of claim 8, further comprising administering the composition concurrently with the substance.

11. The method of claim 8, wherein the cGMP is in solid, semi-solid, or liquid form.

12. The method of claim 8, wherein the cGMP is selected from the group consisting of. cyclic guanosine 3′, 5′-cyclic monophosphate; guanosine 3′, 5′-monophosphate; 3′, 5′-GMP; cGMP; guanosine 3′, 5′-(hydrogen phosphate); guanosine 3′, 5′-cyclic monophosphate; and guanosine 3′, 5′-cyclic phosphate.

13. The method of claim 8 wherein the carrier is selected from the group of pharmaceutically acceptable carriers consisting of tablets, capsules, solutions, emulsions and suspensions.

14. A method of enhancing the bioavailability of an orally ingested pharmaceutical, vitamin, or nutritional supplement, comprising administering to a subject a composition comprising cGMP in an amount and form effective for promoting the contact of the cGMP with the small intestine, prolonging residence time, and promoting absorption/bioavailability of the pharmaceutical, vitamin, or nutritional supplement.

15. The method of claim 14, wherein the composition is in the form of a solid, a solution, an emulsion or a dispersion.

16. The method of claim 14, wherein the composition is in admixture with an organic or inorganic carrier or excipient.

17. The method of claim 16, wherein the composition is compounded with a pharmaceutically acceptable carrier for tablets, capsules, solutions, emulsions, suspensions, and any other form suitable for use.

18. The method of claim 17, wherein the carrier comprises any carrier suitable for use in manufacturing preparations of pharmaceuticals, supplements, or vitamins, in solid, semisolid, or liquid form.

19. The method of claim 16, further comprising at least one of the following ingredients: water, oils, paraffins, powders, granules, syrups, thickeners, detergents, salts, suspending, emulsifying, stabilizing, buffering, preserving, coloring, disintegrating, solubilizing, flavoring and sweetening agents.

20. A composition useful in prolonging the residence time of an administered substance in the small intestine of a subject, comprising:
a. a carrier, and
b. cGMP in an amount and form effective to promote contact of the cGMP with the subject's small intestine, thereby prolonging the residence time of the administered substance to assist in the dissolution, bioavailability and/or increased substance absorption through the small intestine.

21. The composition of claim 20, wherein the administered substance is selected from the group consisting of pharmaceuticals, vitamins, drugs, and supplement agents.

22. The composition of claim 20, wherein the cGMP is in solid, semi-solid, or liquid form.

23. The composition of claim 20, wherein the cGMP is selected from the group consisting of: cyclic guanosine 3′, 5′-cyclic monophosphate; guanosine 3′, 5′-monophosphate; 3′, 5′-GMP; cGMP; guanosine 3′, 5′-(hydrogen phosphate); guanosine 3′, 5′-cyclic monophosphate; and guanosine 3′, 5′-cyclic phosphate.

24. The composition of claim 20 wherein the carrier is selected from the group of pharmaceutically acceptable carriers consisting of tablets, capsules, solutions, emulsions and suspensions.

25. The composition of claim 20 further comprising the administered substance.

26. The composition of claim 25, wherein the administered substance includes one or more of an active pharmaceutical, vitamin, or supplement agent.
